SEQUENCE LISTING (10 sequences)

SEQ ID NO: 1 - RNA, 23 nt, Artificial Sequence
  miRNA hsa-miR-142-3p
uguaguguuuccuacuuuaugga

SEQ ID NO: 2 - RNA, 21 nt, Artificial Sequence
  ath-miR159a
uuuggauugaagggagcucua

SEQ ID NO: 3 - DNA, 49 bp, Artificial Sequence
  RT primer 159aRT
gtcgtatccagtgcagggtccgaggtattcgcactggatacgactagag

SEQ ID NO: 4 - DNA, 17 bp, Artificial Sequence
  Probe 159aP
ctggatacgactagagc

SEQ ID NO: 5 - DNA, 21 bp, Artificial Sequence
  qPCR 5p primer 159aF
gccgtttggattgaagggagc

SEQ ID NO: 6 - DNA, 16 bp, Artificial Sequence
  qPCR 3p primer UPR
gtgcagggtccgaggt

SEQ ID NO: 7 - DNA, 51 bp, Artificial Sequence
  RT primer 142-3pRT
gtcgtatccagtgcagggtccgaggtattcgcactggatacgactccataa

SEQ ID NO: 8 - DNA, 18 bp, Artificial Sequence
  Probe 142-3pP
ctggatacgactccataa

SEQ ID NO: 9 - DNA, 24 bp, Artificial Sequence
  qPCR 5p primer 142-3pF
cgccgctgtagtgtttcctacttt

SEQ ID NO: 10 - DNA, 16 bp, Artificial Sequence
  qPCR 3p primer UPR
gtgcagggtccgaggt

(19) United States
(12) Patent Application Publication
(10) Pub. No.: US 2021/0238682 A1
(43) Pub. Date: Aug. 5, 2021

(54) COMPOSITIONS AND METHODS FOR DONOR SELECTION AND PROGNOSIS OF ACUTE GRAFT-VERSUS-HOST DISEASE

(71) Applicant: DUKE UNIVERSITY, DURHAM, NC (US)

(72) Inventors: YOU-WEN HE, DURHAM, NC (US); YU WANG, JINZHOU, LIAONING (CN); NELSON CHAO, DURHAM, NC (US)

(73) Assignee: DUKE UNIVERSITY, DURHAM, NC (US)

(21) Appl. No.: 17/048,912

(22) PCT Filed: Apr. 19, 2019
(86) PCT No.: PCT/US2019/028326

§ 371 Date: Oct. 19, 2020

Related U.S. Application Data
(60) Provisional application No. 62/659,807, filed on Apr. 19, 2018.

(51) Int. Cl. C12Q 1/6883; A61K 35/28

(52) U.S. Cl. CPC C12Q 1/6883; C12Q 2600/178; C12Q 2600/118; A61K 35/28

(57) ABSTRACT

Provided are compositions and methods for donor selection and prognosis of acute graft-versus-host disease. Provided is a method, which comprises obtaining a first biological sample from a first subject at risk of GVHD; detecting a level of miR-142-3p in the sample; and determining a risk of, prognosis of, or diagnosis of GVHD in the first subject. Provided also is a method for selecting a stem cell transplant donor, which comprises obtaining a first biological sample from a first subject and a second biological sample from a second subject, detecting a level of miR-142-3p in the first biological sample and the second biological sample; determining a ratio of the level of miR-142-3p in the second biological sample to the level of miR-142-3p in the first biological sample; determining the likelihood of the first subject will develop GVHD based on the ratio; and selecting a transplant donor based on the ratio.

### CROSS REFERENCED APPLICATIONS

This application claims the benefit of U.S. Provisional Application No. 62/659,807, filed on Apr. 19, 2018, which is incorporated herein by reference in its entirety.

### STATEMENT AS TO FEDERALLY SPONSORED RESEARCH

This invention was made with Government support under Federal Grant No. AI119707 awarded by the NIH/NIAID. The Federal Government has certain rights to this invention.

### SEQUENCE LISTING

The instant application contains a sequence listing which has been submitted in ASCII format via EFS-Web and is hereby incorporated by reference in its entirety. Said ASCII copy, created on Apr. 19, 2019, is named DU6176PCT_Seq_Listing_ST25.txt and is 2,186 bytes in size.

### FIELD

Provided are compositions and methods for donor selection, diagnosis, prognosis and determining risk of acute graft-versus-host disease in a subject.

### BACKGROUND

Allogeneic bone marrow stem cell transplantation (BMT) is an effective therapeutic method for the treatment of leukemia as well as other non-malignant diseases. However, engrafted T cells may attack many organs in the recipient, thus inducing acute graft-versus-host disease (aGVHD) within 100 days after BMT. Human leukocyte antigen (HLA) is currently the major consideration to match with a donor with a recipient for bone marrow or cord blood transplant. A close HLA match between donor and recipient lowers the chance the recipient will develop aGVHD. However, other biomarkers are needed for determining risk associated with development of GVHD.

### SUMMARY

The present disclosure provides, in part, compositions and methods for donor selection and prognosis of acute graft-versus-host disease in a subject. Provided are methods comprising (a) obtaining a first biological sample from a first subject at risk of GVHD, and (b) detecting a level of miR-142-3p in the sample. In some aspects, the sample has a decreased level of miR-142-3p as compared to a second biological sample obtained from a second subject not at risk of GVHD. In some aspects, the first subject is in need of a bone marrow stem cell transplant. Some aspects comprise comparing the level of miR-142-3p in the sample obtained from the first subject with a level of miR-142-3p obtained from the second subject. Some aspects comprise determining a risk of, prognosis of, or diagnosis of GVHD in the first subject. Some aspects comprising performing a stem cell transplant on the first subject.

Also provided are methods for selecting a stem cell transplant donor, comprising: (a) obtaining a first biological sample from a first subject and a second biological sample from a second subject, (b) detecting a level of miR-142-3p in the first biological sample and the second biological sample; (c) determining a ratio of (i) the level of miR-142-3p in the second biological sample to (ii) the level of miR-142-3p in the first biological sample; and (d) determining the likelihood of the first subject will develop GVHD based on the ratio. Some aspects comprise selecting a transplant donor based on the ratio.

Some aspects comprise not selecting a transplant donor based on the ratio. Some aspects comprise transplanting stem cells from the second subject to the first subject if the ratio of (i):(ii) is at least about 2:1. In some aspects the ratio is about 2.5:1, about 3:1, about 3.5:1, about 4:1, about 4.5:1, or about 5:1.

In some aspects, the methods comprise obtaining one or more additional biological samples from one or more additional subjects, wherein the ratio of (i) the level of miR-142-3p in the second biological sample to (ii) the level of miR-142-3p in the first biological sample is higher than the ratio of (i) the level of miR-142-3p in the one or more additional biological samples to (ii) the level of miR-142-3p in the first biological sample; and transplanting stem cells from the second subject to the first subject.

In some aspects the first subject and the second subject are HLA matched. In other aspects, the first subject and the second subject are HLA mismatched.

Also provided are methods for determining whether a subject will develop GVHD, comprising: (a) obtaining a first biological sample from a first subject that has undergone a stem cell transplant; (b) detecting a level of miR-142-3p in the first biological sample; (c) obtaining a second biological sample from the first subject, wherein the second biological sample is obtained from the subject after the first biological sample is obtained from the subject; (d) determining a level of miR-142-3p in the second biological sample; (e) determining a ratio of (i) the level of miR-142-3p in the second biological sample to (ii) the level of miR-142-3p in the first biological sample; and (f) determining the likelihood the first subject will develop GVHD based on the ratio. Some aspects comprise treating the subject for GVHD. In some aspects the treatment comprises administering one or more of an immunosuppressive drug, a chemotherapy, a steroid, an antifungal agent, and antiviral agent, or an antibiotic. In some aspects, the immunosuppressive drug comprises one or more of cyclosporine, tacrolimus, methotrexate, sirolimus, mycophenolic acid, and rutiximab; the chemotherapy comprises methotrexate; the steroid comprises prednisone or methylprednisolone; the antifungal agent comprises posaconazole; the antiviral agent comprises acyclovir or valacyclovir; and the antibiotic comprises sulfamethoxazole.

Also provided are methods for determining the efficacy of a GVHD treatment, comprising: (a) obtaining a first biological sample from a first subject that has been treated with an anti-GVHD therapy; (b) detecting a level of miR-142-3p in the first biological sample; (c) obtaining a second biological sample from the first subject, wherein the second biological sample is obtained from the subject after the first biological sample is obtained from the subject; (d) determining a level of miR-142-3p in the second biological sample; (e) determining a ratio of (i) the level of miR-142-3p in the second biological sample to (ii) the level of miR-142-3p in the first biological sample; and (f) determining the efficacy of the anti-GVHD therapy based on the ratio. Some aspects comprise altering treatment of the GVHD if the ratio is below about 2:1. In some aspects, the anti-GVHD therapy comprises one or more of an immunosuppressive drug, a chemotherapy, a steroid, an antifungal agent, and antiviral agent, or an antibiotic. In some aspects, the immunosuppressive drug comprises one or more of cyclosporine, tacrolimus, methotrexate, sirolimus, mycophenolic acid, and rutiximab; the chemotherapy comprises methotrexate; the steroid comprises prednisone or methylprednisolone; the antifungal agent comprises posaconazole; the antiviral agent comprises acyclovir or valacyclovir; and the antibiotic comprises sulfamethoxazole.

In some aspects, the first subject and/or the second subject is a mammal, such as a human.

In some aspects, the first biological sample and/or the second biological sample is selected from the group consisting of tissues, cells, biopsies, blood, lymph, serum, plasma, urine, saliva, mucus, and tears. In particular aspects, the sample comprises blood or plasma.

Also provided are compositions for conducting the provided methods. In some aspects, the compositions comprise a probe array for determining a level of miR-142-3p in a biological sample, the array comprising of a plurality of probes that hybridizes to the miR-142-3p. In some aspects, the compositions comprise a kit for determining a level of miR-142-3p in a biological sample, comprising the probe array of and instructions for carrying out the determination of miR-142-3p expression level in the biological sample. In some aspects, the probe array comprises a solid support with the plurality of probes attached thereto.

Also provided are methods of determining the risk of, prognosis of, and/or diagnosis of GVHD in a subject comprising, consisting of, or consisting essentially of quantifying the amount of at least one biomarker present in a biological sample derived from the subject, where in the biomarker comprises, consists of, or consists essentially of an miRNA associated with GVHD.

Some aspects comprise methods of selecting a donor for transplant recipient comprising, consisting of, or consisting essentially of: (a) obtaining a biological sample from the donor; (b) determining the expression level of one or more miRNA biomarkers that are associated with GVHD in the biological sample; (c) comparing the expression level of the one or more miRNA biomarkers in the biological samples with that of a control, where miRNA expression that is lower than the control indicates a poor match; and (d) not selecting the donor for transplant.

In some aspects, the methods further comprises matching the donor-recipient HLA matching where (a) donor-recipient HLA mismatch and low miRNA expression results in not selecting the donor for transplant and (b) donor-recipient HLA match and low miRNA expression results in not selecting the donor for transplant.

Also provided are methods determining the prognosis of a subject developing, or having already developed, GVHD after a transplantation event comprising, consisting of, or consisting essentially of: (a) obtaining a biological sample from a subject; (b) determining the expression level of one or more miRNA biomarkers that are associated with GVHD in the biological sample; comparing the expression level of the miRNA biomarkers in the biological sample with that of a control, where miRNA expression that is lower than the control indicates a poor prognosis and (c) administering an appropriate anti-GVHD therapy or altering an already administered anti-GVHD therapy, if one or more of the biomarkers are expressed at low levels.

Also provided are methods for determining the efficacy of an GVHD treatment regime in a subject comprising, consisting of, or consisting essentially of: (a) determining a baseline value for the expression of one or more miRNA biomarkers associated with GVHD; (b) administering to the subject an anti-GVHD therapy regime; and (c) redetermining the expression levels of one or more biomarkers in the subject, where in observed increases in one or more of the miRNA biomarker expression levels is correlated with the efficacy of the therapeutic regimen.

### BRIEF DESCRIPTION OF THE DRAWINGS

FIGS. 1 A-F relate to profiling for candidate miRNAs using a high throughput platform. FIG. 1A depicts a schematic of RNA extraction using an anti-miRNA bead capture method; FIG. 1B depicts a schematic of a Taqman miRNA realtime PCR assay; FIG. 1C depicts a schematic of a Taqman microRNA openarray chip; FIG. 1D provides a Taqman openarray profiling result of plasma microRNAs from before onset samples, which were drawn 1-3 days before onset of aGVHD (n=19) and time point matched non-GVHD (n=23) from Duke Hospital; FIG. 1E provides the relative expression level of miR-142-3p in the plasma from non-GVHD (n=23) and aGVHD (n=19) patients, with significance determined by a two-tailed Mann-Whitney test (**p<0.01); FIG. 1F provides an ROC analysis of plasma miR-142-3p.

FIGS. 2A and 2B depict graphs showing that plasma miR-142-3p ratio is associated with aGVHD development. FIG. 2A depicts the ratio of plasma miR-142-3p level between donor and recipient in aGVHD (n=53) and non-GVHD patients (n=56); FIG. 2B depicts the ratio of plasma miR-142-3p level between day 28 and day 0 from the same aGVHD (n=52) or non-GVHD (n=56) patient; significance in both FIGS. 2A and 2B was determined by two-tailed Student t test (***p<0.001).

### DETAILED DESCRIPTION

Technical and scientific terms used herein have the meanings commonly understood by one of ordinary skill in the art to which the present subject matter pertains, unless otherwise defined. Reference is made herein to various methodologies known to those of ordinary skill in the art. Any suitable materials and/or methods known to those of ordinary skill in the art can be utilized in carrying out aspects provided herein. However, specific materials and methods are described. Materials, reagents and the like to which reference is made in the following description and examples are obtainable from commercial sources, unless otherwise noted. Publications and other materials setting forth such known methodologies to which reference is made are incorporated herein by reference in their entireties as though set forth in full.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the disclosed subject matter. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The use herein of the terms “including,” “comprising,” or “having,” and variations thereof, is meant to encompass the elements listed thereafter and equivalents thereof as well as additional elements. Aspects set forth as “including,” “comprising” or “having” certain elements are also contemplated as “consisting essentially of” and “consisting of” those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “miRNA” or “miR” or “microRNA” refers to a non-coding RNA between 10 and 30 nucleotides in length which hybridizes to and regulates the expression of a coding RNA (see, Zeng and Cullen, RNA, 9(1): 112-123, 2003; Kidner and Martienssen Trends Genet, 19(1): 13-6, 2003; Dennis C, Nature, 420(6917):732, 2002; Couzin J, Science 298(5602):2296-7, 2002, each of which is incorporated by reference herein). A 10 to 30 nucleotide miRNA molecule can be obtained from a miRNA precursor through natural processing means (e.g., using intact cells or cell lysates) or by synthetic processing routes (e.g., using isolated processing enzymes, such as isolated Dicer, Argonaut, or RNAse III). It is understood that the 10 to 30 nucleotide RNA molecule can also be produced directly or by biological or chemical synthesis, without having been processed from a miR precursor.

Included within this definition is natural miRNA molecules, pre-miRNA, pri-miRNA, miRNA molecules identical in nucleic acid sequence to the natural forms as well as nucleic acid sequences, where in one or more nucleic acids has been replaced or is represented by one or more DNA nucleotides and/or nucleic acid analogue. miRNA molecules in the present specification are occasionally referred to as a nucleic acid molecule(s) encoding a miRNA or simply nucleic acid molecule(s).

As used herein, the term “biomarker” refers to a naturally occurring biological molecule present in a subject at varying concentrations useful in predicting the risk or incidence of a disease or a condition, such as GVHD. For example, the biomarker can be an miRNA present in higher or lower amounts in a subject at risk for GVHD. The biomarker can include nucleic acids, ribonucleic acids, or a polypeptide used as an indicator or marker for GVHD in a cell, tissue or subject. In certain aspects, the biomarker is an miRNA, such as miR-142-3p.

As used herein, the terms “acute graft-versus-host disease,” “aGVHD,” and “GVHD” are used interchangeable and refer to the acute or fulminant form of GVHD that is normally observed within the first 100 days post-transplantation. GVDH is discussed, for instance in Nassereddine, Anticancer Research, 37(4): 1547-1555 (2017).

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Those in need of treatment include those already affected by a disease or disorder or undesired physiological condition as well as those in which the disease or disorder or undesired physiological condition is to be prevented. Treatment/therapy regimens will be different for each subject depending on several factors, including age/health of a subject, stage of disease, etc. and can be readily determined by an attending physician. For example, possible treatment/therapies include, but are not limited to, administration of immunosuppressive drugs, selective depletion of alloreactive T lymphocytes, use of monoclonal antibodies (e.g., anti-CD3, anti-CDS, IL-2 antibodies) etc. As used herein, the term “prophylactic treatments” refer to those therapies that are used to prevent the occurrence of a condition such as GVHD from happening. Suitable prophylactic treatments may include prophylactic treatment with immunosuppressive drugs, use of umbilical cord blood as the source of donor cells, closer HLA matching between donor and patients, etc.

The term “effective amount” or “therapeutically effective amount” refers to an amount sufficient to effect beneficial or desirable biological and/or clinical results.

As used herein, the term “subject” and “patient” are used interchangeably and refer to both human and nonhuman animals. The term “nonhuman animals” includes all vertebrates, e.g., mammals and non-mammals, such as nonhuman primates, sheep, dog, cat, horse, cow, chickens, amphibians, reptiles, and the like. In some aspects, the subject is a human. In certain aspects, the subject is a human at risk for GVHD. The subject can be, but is not limited to, a transplant recipient, a transplant donor, a potential transplant recipient, or a potential transplant donor.

The term “biological sample” as used herein includes, but is not limited to, a sample containing tissues, cells, and/or biological fluids isolated from a subject. Examples of biological samples include tissues, cells, biopsies, blood, lymph, serum, plasma, urine, saliva, mucus and tears. In some aspects, the biological sample is a blood sample (such as a plasma sample). A biological sample may be obtained directly from a subject (e.g., by blood or tissue sampling) or from a third party (e.g., received from an intermediary, such as a healthcare provider or lab technician).

As used herein, the term “transplantation event” refers to the act of transplanting from a donor to a recipient an organ, blood, bone marrow or other biological material. In some aspects, the transplantation event comprises a bone marrow transfer (BMT).

### I. Introduction

Any mircoRNA having an association with GVHD may be assessed according to the present methods. Particular aspects relate to assessing the risk of developing GVHD based on a miR-142-3p level in a biological sample. miR-142-3p is a microRNA believed to play a role in hematopoietic development, and which has an exemplary sequence UGUAGUGUUUCCUACUUUAUGGA (SEQ ID NO: 1).

### II. Graft-Versus-Host Disease

GVHD is a potentially serious complication of allogeneic stem cell transplantation. In some aspects, GVHD results from a subject receiving stem cells from a donor or donated umbilical cord blood. T cells from the donor may attack healthy cells, tissues, or organs in the recipient, thus impairing the function or causing failure of the cell, tissue, or organ. Without being bound by theory, it is believed that GVHD is caused by one or more of administering to a recipient an immuno-competent graft with viable and functional immune cells; performing a transplant in a recipient that is immunologically different from a donor (i.e., histo-incompatibility); and performing a transplant in an immunocompromised recipient.

In some aspects, GVHD is associated with damage to one or more of the liver, skin, mucosa, and gastrointestinal tract. In some aspects, GVHD impacts one or both of the immune system (e.g., the hematopoietic system, such as the bone marrow and/or the thymus) and the lungs (e.g., in the form of immune-mediated pneumonitis). Symptoms associated with GVHD include, but are not limited to, one or more of intestinal inflammation, sloughing of the mucosal membrane, diarrhea, abdominal pain, nausea, vomiting, high bilirubin levels, red maculopapular rash, and mucosal damage.

Subjects undergoing an allogeneic stem cell transplant or umbilical cord blood transplant are at risk of developing GVHD. Other risk factors include degree of human leukocyte antigen (HLA) disparity (i.e., HLA mismatch vs. HLA match (see (Kanda, Int. J. Hematol., 98(3): 300-308 (2013)))) between a donor and a recipient, donor and recipient gender disparity (e.g., female donor to male recipient or male donor to female recipient), intensity of transplant conditioning regimen, GVHD prophylactic regimen used, source of graft (e.g., peripheral blood, bone marrow, or umbilical cord blood), age of the recipient, cytomegalovirus status of the donor and the recipient, donor Epstein-Barr virus sensitivity, the presence of a sterile environment (e.g., gut decontamination), and particular HLA haplotype. HLA can be a consideration to match a recipient with a donor for bone marrow or cord blood transplant. A close HLA match between donor and recipient lowers the chance to develop aGVHD. HLA matching or mismatching can involve HLA-A, -B, -C, -DRB1, -DQB1, and -DPB1 loci.

Several systems for grading acute GVHD have been developed. Two such systems are the Glucksberg grade (I-IV) and the International Bone Marrow Transplant Registry (IBMTR) grading system (A-D) (Glucksberg, Transplantation, 18(4): 295-304 (1974); Rowlings, Br. J. Haemotol., 97: 855-864 (1997)) The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade. Grade I(A) GVHD is characterized as mild disease, grade II(B) GVHD as moderate, grade III(C) as severe, and grade IV(D) life-threatening.

The IBMTR grading system defines the severity of acute GVHD as follows: Grade A-Stage 1 skin involvement alone (maculopapular rash over <25 percent of the body) with no liver or gastrointestinal involvement; Grade B-Stage 2 skin involvement; Stage 1 to 2 gut or liver involvement; Grade C-Stage 3 involvement of any organ system (generalized erythroderma; bilirubin 6.1 to 15.0 mg/dL; diarrhea 1500 to 2000 mL/day); Grade D-Stage 4 involvement of any organ system (generalized erythroderma with bullous formation; bilirubin >15 mg/dL; diarrhea >2000 mL/day or pain or ileus).

III. Methods of Detecting miR-142-3p

Provided are methods of detecting miRNA, such as miR-142-3p. In some aspects, the miRNA is detected in a biological sample, e.g., a biological sample obtained from a subject. In some aspects, the miRNA is extracellular miRNA. In some aspects the miRNA is circulating miRNA, e.g., miRNA circulating in the blood.

Some aspects involve obtaining more than one sample, such as two or more samples, such as three samples, four samples, or more from subjects. In some aspects, the samples are obtained from the same subject. In some aspects, the samples are obtained from different subjects. Some aspects comprise conducting a relative comparison of expression in the presence or absence of at least one nucleic acid and/or of the level of expression of the at least one nucleic acid between the two or more samples. Alternatively, a single sample may be compared against a “standardized” sample, such a sample comprising material or data from several samples, preferably also from several individuals.

In some aspects, one or more sample preparation operations are performed upon the sample before analyzing the sample. Such sample preparation operations include, but are not limited to, such manipulations as concentration, suspension, extraction of intracellular material, e.g., nucleic acids from tissue/whole cell samples and the like, amplification of nucleic acids, fragmentation, transcription, labelling and/or extension reactions.

Nucleic acids, especially RNA and specifically miRNA can be isolated using any techniques known in the art, such as phenol-based extraction and silica matrix or glass fiber filter (GFF)-based binding. Phenol-based reagents contain a combination of denaturants and RNase inhibitors for cell and tissue disruption and subsequent separation of RNA from contaminants. Phenol-based isolation procedures can recover RNA species in the 10-200-nucleotide range e.g., miRNAs, 5S rRNA, 5.8S rRNA, and Ul snRNA. If a sample of “total” RNA is purified by the popular silica matrix column or GFF procedure, it may be depleted in small RNAs. Extraction procedures such as those using Trizol or TriReagent, however will purify all RNAs, large and small, and may be used for isolating total RNA from biological samples that will contain miRNAs/siRNAs.

Any method required for the processing of a sample prior to detection by any of the methods noted herein falls within the scope of the present disclosure.

It is within the general scope of the present disclosure to provide methods for the detection of miRNA. Some aspects relate to the detection of the miRNA sequences as described in the plots and graphs of the figures contained herein. As used herein, the term “detect” or “determine the presence of” refers to the qualitative measurement of undetectable, low, normal, or high concentrations of one or more biomarkers such as, for example, nucleic acids, ribonucleic acids, or polypeptides and other biological molecules.

Detection may include 1) detection in the sense of presence versus absence of one or more miRNAs as well as 2) the registration/quantification of the level or degree of expression of one or more miRNAs, depending on the method of detection employed. The term “quantify” or “quantification” may be used interchangeable, and refer to a process of determining the quantity or abundance of a substance in a sample (e.g., a biomarker), whether relative or absolute. For example, quantification may be determined by methods including but not limited to, micro-array analysis, qRT-PCR, band intensity on a Northern or Western blot, or by various other methods in the art.

The detection of one or more nucleic acid molecules allows for the classification, diagnosis and prognosis of a condition such as GVHD. The classification of such conditions is of relevance both medically and scientifically and may provide important information useful for the diagnosis, prognosis and treatment of the condition. The diagnosis of a condition such as a GVHD is the affirmation of the presence of the disease based, as is the object of the present disclosure, on the expression of at least one miRNA herein also referred to as a nucleic acid molecule. Prognosis is the estimate or prediction of the probable outcome of a condition such as a GVHD and the prognosis of such is greatly facilitated by increasing the amount of information on the particular condition.

Any method of detection falls within the general scope of the present disclosure. The detection methods may be generic for the detection of nucleic acids (e.g., RNA), or be optimized for the detection of small RNA species (e.g., mature and/or precursor miRNAs) or be designed for the detection of miRNA species. The detection methods may be directed towards the scoring of a presence or absence of one or more nucleic acid molecules or may be useful in the detection of expression levels.

The detection methods can be divided into two categories herein referred to as in situ methods or screening methods. The term in situ method refers to the detection of nucleic acid molecules in a sample where in the structure of the sample has been preserved. This may thus be a biopsy where in the structure of the tissue is preserved. In situ methods are generally histological i.e. microscopic in nature and include but are not limited to methods such as: in situ hybridization techniques and in situ PCR methods.

Screening methods generally employ techniques of molecular biology and usually involve the preparation of the sample material in order to access the nucleic acid molecules to be detected. Screening methods include, but are not limited to methods such as: Array systems, affinity matrices, Northern blotting and PCR techniques, such as real-time quantitative RT-PCR.

One aspect of the present disclosure is to provide a probe which can be used for the detection of a nucleic acid molecule as defined herein. A probe as defined herein is a specific sequence of a nucleic acid used to detect nucleic acids by hybridization. A nucleic acid can include a natural or synthetic nucleic acid such as DNA, RNA, LNA or PNA. A probe may be labeled, tagged or immobilized or otherwise modified according to the requirements of the detection method chosen. A label or a tag can be used for identification a compound to which it is associated. Some aspects employ probes that are labeled or tagged by any means in the art such as, but not limited to: radioactive labeling, fluorescent labeling and enzymatic labeling. Furthermore the probe, labeled or not, may be immobilized to facilitate detection according to the detection method of choice.

In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules as herein described. Sample cells can be treated to increase their permeability to allow the probe to enter the cells, the probe can be added to the treated cells, allowed to hybridize at pertinent temperature, and then excess probe can be washed away. A complementary probe can be labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the miRNAs mentioned herein.

In situ PCR is the PCR based amplification of the target nucleic acid sequences prior to ISH. For detection of RNA, an intracellular reverse transcription (RT) step can be introduced to generate complementary DNA from RNA templates prior to in situ PCR. This enables detection of low copy RNA sequences.

Prior to in situ PCR, cells or tissue samples can be fixed and permeabilized to preserve morphology and permit access of the PCR reagents to the intracellular sequences to be amplified. PCR amplification of target sequences can then be performed either in intact cells held in suspension or directly in cytocentrifuge preparations or tissue sections on glass slides. In the former approach, fixed cells suspended in the PCR reaction mixture can be thermally cycled using conventional thermal cyclers. After PCR the cells can be cytocentrifugated onto glass slides with visualization of intracellular PCR products by ISH or immunohistochemistry. In situ PCR on glass slides can be performed by overlaying the samples with the PCR mixture under a coverslip which can be sealed to prevent evaporation of the reaction mixture. Thermal cycling can be achieved by placing the glass slides either directly on top of the heating block of a conventional or specially designed thermal cycler or by using thermal cycling ovens. Detection of intracellular PCR-products can be achieved by various techniques, such as indirect in situ PCR by ISH with PCR-product specific probes, or by direct in situ PCR without ISH through direct detection of labeled nucleotides (e.g. digoxigenin-11-dUTP, fluorescein-dUTP, H-CTP or biotin-16-dUTP) which have been incorporated into the PCR products during thermal cycling.

In some aspects, detection is via a microarray. A microarray is a microscopic, ordered array of nucleic acids, proteins, small molecules, cells or other substances that enables parallel analysis of complex biochemical samples. A DNA microarray has different nucleic acid probes, known as capture probes that are chemically attached to a solid substrate, which can be a microchip, a glass slide or a microsphere-sized bead. Microarrays can be used e.g. to measure the expression levels of large numbers of mRNAs/miRNAs simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

Some aspects involve the use of microarrays for the expression profiling of miRNAs in conditions such as GVHD. By way of example, RNA can be extracted from a cell or tissue sample, and the small RNAs (18-26-nucleotide RNAs) can be size-selected from total RNA using denaturing polyacrylamide gel electrophoresis (PAGE). Then oligonucleotide linkers can be attached to the 5′ and 3′ ends of the small RNAs and the resulting ligation products can be used as templates for an RT-PCR reaction with 10 cycles of amplification. The sense strand PCR primer can have a Cy3 fluorophore attached to its 5′ end, thereby fluorescently labelling the sense strand of the PCR product. The PCR product can be denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot can then be evaluated in terms of the number of copies of a particular miRNA, using a number of positive and negative controls and array data normalization methods, which will result in assessment of the level of expression of a particular miRNA.

Several types of microarrays can be employed such as spotted oligonucleotide microarrays, pre-fabricated oligonucleotide microarrays or spotted long oligonucleotide arrays.

In spotted oligonucleotide microarrays the capture probes can be oligonucleotides complementary to miRNA sequences. This type of array can be hybridized with amplified PCR products of size-selected small RNAs from two samples to be compared that are labelled with two different fluorophores. Alternatively, total RNA containing the small RNA fraction (including the miRNAs) can be extracted from the abovementioned two samples and used directly without size-selection of small RNAs, and 3′ end labeled using T4 RNA ligase and short RNA linkers labelled with two different fluorophores. The samples can be mixed and hybridized to one single microarray that can be scanned, allowing the visualization of up-regulated and down-regulated miRNA genes In some aspects, a universal reference can be used, comprising of a large set of fluorophore-labelled oligonucleotides, complementary to the array capture probes.

In pre-fabricated oligonucleotide microarrays or single-channel microarrays, the probes can be designed to match the sequences of known or predicted miRNAs. Commercially available designs from companies such as Affymetrix or Agilent can cover complete genomes. These microarrays give estimations of the absolute value of gene expression and therefore the comparison of two conditions cane use two separate microarrays.

Spotted long oligonucleotide arrays are composed of 50 to 70-mer oligonucleotide capture probes, and can be produced by either ink-jet or robotic printing. Short Oligonucleotide Arrays are composed of 20-25-mer oligonucleotide probes, and can be produced by photolithographic synthesis (Affymetrix) or by robotic printing. More recently, Maskless Array Synthesis from NimbleGen Systems has combined flexibility with large numbers of probes. Arrays can contain up to 390,000 spots, from a custom array design.

The terms “PCR reaction”, “PCR amplification”, “PCR”, “pre-PCR”, “Q-PCR”, “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Some aspects involve using the nucleic acid sequence based amplification and Q Beta Replicase systems. The products formed by said amplification reaction may or may not be monitored in real time, or only after the reaction as an end-point measurement.

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This can be used to determine whether a genetic sequence is present or not, and if it is present the number of copies in the sample. The process can be used to amplify DNA samples, using thermal cycling and a thermostable DNA polymerase.

Polymerase chain reactions can be performed with agarose gel electrophoresis, the use of SYBR Green, a double stranded DNA dye, and/or the fluorescent reporter probe. In agarose gel electrophoresis, an unknown sample and a known sample can be prepared with a known concentration of a similarly sized section of target DNA for amplification. Both reactions can be run for the same length of time in identical conditions (preferably using the same primers, or at least primers of similar annealing temperatures). Agarose gel electrophoresis can be used to separate the products of the reaction from their original DNA and spare primers. The relative quantities of the known and unknown samples can be measured to determine the quantity of the unknown.

SYBR Green dye is a DNA binding dye binds all newly synthesized double stranded (ds)DNA and an increase in fluorescence intensity is measured, thus allowing initial concentrations to be determined. A reaction can be run with the addition of fluorescent dsDNA dye, and the levels of fluorescence can be monitored, with the dye fluorescing when bound to the dsDNA. With reference to a standard sample or a standard curve, the dsDNA concentration in the PCR can be determined.

A fluorescent reporter probe uses a sequence-specific nucleic acid based probe so as to quantify the probe sequence and not all double stranded DNA. It can be carried out with DNA based probes with a fluorescent reporter and a quencher held in adjacent positions, so-called dual-labelled probes. The close proximity of the reporter to the quencher prevents its fluorescence; it upon the breakdown of the probe the fluorescence can be detected. This process depends on the 5′ to 3′ exonuclease activity of the polymerase involved. The real-time quantitative PCR reaction can be prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerization continues it reaches the probe bound to its complementary sequence, which is then hydro lysed due to the 5′-3′ exonuclease activity of the polymerase thereby separating the fluorescent reporter and the quencher molecules. This results in an increase in fluorescence, which can be detected. During thermal cycling of the real-time PCR reaction, the increase in fluorescence, as released from the hydrolysed dual-labelled probe in each PCR cycle can be monitored, which allows accurate determination of the final, and so initial, quantities of DNA.

Any method of PCR that can determine the expression of a nucleic acid molecule as defined herein falls within the scope of the present disclosure. Some aspects include the real-time quantitative RT-PCR method, based on the use of either SYBR Green dye or a dual-labelled probe for the detection and quantification of nucleic acids according to the herein described.

An aspect of the present disclosure includes the detection of the nucleic acid molecules herein disclosed by techniques such as Northern blot analysis.

Yet another aspect of the present disclosure includes a kit for predicting GVHD in a subject, the kit comprising: primers for reverse transcribing one or more miRNA biomarkers for GVHD in a biological sample derived from a subject, where in the miRNA biomarkers comprises miR-142-3p and instructions for quantifying the expression level of the miRNA biomarkers in the biological sample and for predicting the subject as having an increased risk for development of a GVHD if the expression level of the miRNA biomarker is lower in the biological sample derived from the subject compared to a reference control.

An aspect of the present disclosure includes a kit for diagnosing GVHD in a subject, the kit comprising: primers for reverse transcribing one or more miRNA biomarkers for GVHD in a biological sample derived from a subject, where in the miRNA biomarkers consist of miR-142-3p and instructions for quantifying the expression level of the miRNA biomarkers in the biological sample and for diagnosing the subject as having GVHD if the expression level of the miRNA biomarkers is lower in the biological sample derived from the subject compared to a reference control.

An aspect of the present disclosure includes a kit for determining the prognosis of a subject developing, or having already developed, GVHD, the kit comprising: primers for reverse transcribing one or more miRNA biomarkers for GVHD in a biological sample derived from a subject, where in the miRNA biomarkers biomarkers consist of miR-423, miR-142-3p and instructions for quantifying the expression level of the miRNA biomarkers in the biological sample and for identifying the subject as having a poor chance of survival of a GVHD if the expression level of the miRNA biomarkers is lower in the biological sample derived from the subject compared to a reference control. An aspect of the present disclosure includes a kit for determining the prognosis of a subject developing, or having already developed, GVHD, the kit comprising: primers for reverse transcribing one or more miRNA biomarkers for GVHD in a biological sample derived from a subject, where in the miRNA biomarkers consist of miR-142-3p and instructions for quantifying the expression level of the miRNA biomarkers in the biological sample and for identifying the subject as having a poor chance of survival of a GVHD if the expression level of the miRNA biomarkers is lower in the biological sample derived from the subject compared to a reference control.

Yet another aspect of the present disclosure provides a composition of matter comprising, consisting of, or consisting essentially of: (a) a probe array for determining an miRNA level in a sample, the array comprising of a plurality of probes that hybridizes to one or more miRNAs that are associated with GVHD; or (b) a kit for determining an miRNA level in a sample, comprising the probe array of and instructions for carrying out the determination of miRNA expression level in the sample. In some aspects, the probe array of further comprises a solid support with the plurality of probes attached thereto.

### IV. Risk Determination

Surprisingly, it was determined that the risk of GVHD can be evaluated based on miR-142-3p levels (e.g., expression levels) in a biological sample obtained from a subject. Such miR-142-3p levels can also be used to diagnose GVHD or monitor the progression of GVHD.

For instance, in some aspects a decreased level of miR-142-3p in a biological sample obtained from a subject is associated or correlated with an increased risk that a subject will develop GVHD. In some aspects, an increased level of miR-142-3p in a donor (or potential donor) is associated or correlated with a decreased risk that a recipient (or potential recipient) will develop GVHD.

In some aspects, miR-142-3p levels from two or more subjects—such as 3, 4, 5, 6, 7, 8, 9, or 10 or more subjects—are compared, and a relative risk of GVHD is determined based on the levels of miR-142-3p.

In some aspects, a risk determination is made based on a ratio (e.g., a log 2 ratio) of (i) miR-142-3p levels in a biological sample obtained from a one subject (e.g., a transplant donor or potential donor) to (ii) miR-142-3p levels in a biological sample obtained from a different subject (e.g., a transplant recipient or potential recipient). Exemplary ratios (e.g., log 2 ratios) include about 0.5:1, about 1:1, about 1.5:1, about 2:1, about 2.5:1, about 3:1, about 3.5:1, about 4:1, about 4.5:1, and about 5:1, such as 0.5:1, 1:1, 1.5:1, 2:1, 2.5:1, 3:1, 3.5:1, 4:1, 4.5:1, and 5:1.

In some aspects, a subject is determined to be at risk of developing GVHD is the ratio is less than about 2:1, less than about 1.5:1, less than about 1:1, or less than about 0.5:1, such as less than 2:1, less than 1.5:1, less than 1:1, or less than 0.5.1.

In some aspects, the risk determination is a relative risk determination. For instance, a donor-recipient pair (or potential donor-potential recipient pair) with a higher donor:recipient miR-142-3p ratio may be at decreased risk of GVHD as compared to a donor-recipient (or potential donor-potential recipient pair) with a lower donor:recipient miR-142-3p ratio. Thus, some aspects comprise detecting miR-142-3p levels in one or more potential recipients and one or more potential donors, and assigning risk of GVHD based on the donor:recipient miR-142-3p ratios between the potential donors and potential recipients. Some aspects comprise selecting a donor and/or recipient based on the determined ratios. Some aspects comprising withholding or delaying a transplant based on the determined ratios.

Some aspects comprise determining a risk that a subject that has undergone a stem cell transplant will develop GVHD. Other aspects comprise determining the efficacy of a GVHD treatment. Such a risk can be determined by detecting changes in miR-142-3p levels over time. For instance, miR-142-3p levels can be detected and compared in biological samples obtained from a subject over a period of about 100 days after the subject received a transplant or a GVHD treatment. Such samples can be obtained daily, weekly, or monthly. Thus, in some aspects a biological sample is obtained daily following receipt of the transplant or GVHD treatment. In some aspects, a biological sample is obtained at least once a week from the subject following receipt of the transplant or GVHD treatment. In some aspects, a biological sample is obtained at least once a month following receipt of the transplant or GVHD treatment. In some aspects, a biological sample is obtained 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days after receipt of the transplant or GVHD treatment. In some aspects, the biological sample is obtained immediately following or less than one day after receipt of the transplant or GVHD treatment.

In some aspects, a risk of developing GVHD or is determined based on a ratio of (i) miR-142-3p levels in a biological sample obtained at a later timepoint to (ii) miR-142-3p levels in a biological sample obtained at an earlier timepoint. In some aspect, a ratio (e.g., log 2 ratio) of less than about 3:1, less than about 2:5.1, less than about 2:1, less than about 1.5:1, less than about 1:1, less than about 0.5:1, or less than about −0.1:1, or less than about −1:1 is indicative of a risk of developing GVHD. For instance, in some aspects a ratio of less than 3:1, less than 2:5.1, less than 2:1, less than 1.5:1, less than 1:1, less than 0.5:1, or less than −0.1:1, or less than −1:1 is indicative of a risk of developing GVHD.

As mentioned, some aspects relate to determining the efficacy of a GVHD treatment based on changes in miR-142-3p levels over time. Thus, in some aspects an increase in miR-142-3p levels over time is indicative of an effective GVHD treatment. Alternatively, in some aspects a decrease in miR-142-3p levels over time is indicative of ineffective GVHD treatment. Some aspects relate to determining the efficacy of a GVHD treatment based on a ratio of (i) miR-142-3p levels in a biological sample obtained at a later timepoint to (ii) miR-142-3p levels in a biological sample obtained at an earlier timepoint. In some aspects, a ratio (e.g., log 2 ratio) of less than about 3:1, less than about 2:5.1, less than about 2:1, less than about 1.5:1, less than about 1:1, less than about 0.5:1, or less than about −0.1:1, or less than about −1:1 is indicative of a risk of continuing or worsening GVHD. For instance, in some aspects a ratio of less than 3:1, less than 2:5.1, less than 2:1, less than 1.5:1, less than 1:1, less than 0.5:1, or less than −0.1:1, or less than −1:1 is indicative of a risk of continuing or worsening GVHD.

In some aspects, risk of developing GVHD or efficacy of a GVHD treatment is evaluated based on detecting miR-142-3p levels in combination with other indicators. Such other indicators include, for example, HLA matching or mismatching, and/or detection of increased or decreased levels of biomarkers such as one or more of miR-423, miR-199a-3p, miR-93*, and miR-377. Thus, in some aspects, HLA mismatching and increased levels of one or more of miR-423, miR-199a-3p, miR-93*, and miR-377 indicate a risk for developing GVHD.

Thus, in addition to or apart from other indicators (such as HLA): (1) low miR-142-3p level in the donor plasma is a risk factor for the donor selection and develop of aGVHD after bone marrow transplant; and/or (2) the plasma miR-142-3p level in the recipient after bone marrow transplant can be used to monitor the development of aGVHD.

Some aspects comprises matching a donor and recipient where (a) donor-recipient HLA mismatch and low miR-142-3p expression results in not selecting the donor for transplant and/or (b) donor-recipient HLA match and low miR-142-3p expression results in not selecting the donor for transplant. Some aspects comprise matching a donor and recipient where (a) donor-recipient HLA mismatch and high miR-142-3p expression results in selecting the donor for transplant and/or (b) donor-recipient HLA match and high miR-142-3p expression results in selecting the donor for transplant.

In some aspects, miR-142-3p expression levels are classified as high or low based on comparison to a control or threshold value. Such a control or threshold value can be determined with reference to levels of miR-142-3p expression in GVHD or non-GVHD subjects, or with reference to a population of GVHD or non-GVHD subjects.

### V. GVHD Treatment

Some aspects comprise treating a subject with or at risk for developing GVHD. In some aspects, subjects diagnosed or prognosed with GVHD are administered a treatment. In some aspects, treatment of a subject may be modified upon a determination of the efficacy of the treatment, or of a determination of risk of progression of GVHD.

Some aspects relate to prophylactic treatments, which refers to therapies that prevent the occurrence of GVHD. Suitable prophylactic treatments may include prophylactic treatment with immunosuppressive drugs, use of umbilical cord blood as the source of donor cells, and pursuing a closer HLA match between a donor and recipient.

Some aspects comprise initiating or administering, monitoring and/or modifying treatment regimens. Non-limiting examples of GVHD treatments include immunosuppressive drugs (e.g., one or more of mycophenolate mofetil, Alemtuzumab [Campath], ATG, Sirolimus, cyclosporine, tacrolimus, methotrexate, sirolimus, mycophenolic acid, and rutiximab); selective depletion of alloreactive T lymphocytes; monoclonal antibodies (e.g., anti-CD3, anti-CDS, and/or IL-2 antibodies); chemotherapy (e.g., methotrexate); steroids (e.g., one or both of prednisone and methylprednisolone); antifungal agents (e.g., posaconazole); antiviral agents (e.g., one or both of acyclovir or valacyclovir); and antibiotics (e.g., sulfamethoxazole). Exemplary treatments for GVHD are set forth in Nassereddine, Anticancer Research, 37(4): 1547-1555 (2017).

The following examples are included as illustrative of the compositions described herein. These examples are in no way intended to limit the scope of the invention. Other aspects of the invention will be apparent to those skilled in the art to which the invention pertains.

### EXAMPLES

### Example 1

### Sample Collection, RNA Extraction, Reverse Transcription, Realtime PCR, and PCR Product Sequencing

A study was conducted using a population consisting of 192 human subjects who underwent allogeneic HCT and 114 corresponding donors from multi-centers. Plasma samples were collected from Duke University Medical Center, Dana Farber Cancer Institute and Blood and Marrow Transplant Clinical Trials Network. The miRNA profiling set consisted of 19 HCT patients who had developed aGVHD and 23 HCT patients who never developed aGVHD (non-GVHD) from Duke University Medical Center. Another cohort of 15 non-GVHD and 15 aGVHD samples before aGVHD onset from Dana Farber Cancer Institute were used to perform independent openarray assay to valid the candidate miRNAs identified from Duke Samples. The miR-142-3p identification set included 6 aGVHD and 6 non-GVHD patients as well as correspondent donors to each recipient from Dana Farber Cancer Institute. The miR-142-3p validation set included of 52 aGVHD and 56 non-GVHD patients as well as correspondent donors to each recipient from Clinical Trials Network. EDTA-anticoagulated blood was drawn from the patient and cell free plasma was isolated from all blood samples using a 2-step centrifugation protocol (2000 rpm for 10 min, 12 000 rpm for 3 min) to prevent contamination by cellular nucleic acids. The diagnosis of aGVHD was based on clinical criteria and histologically confirmed by biopsy in the target organs. aGVHD was graded based on the severity of involvement of the target organs.

Total RNAs were extracted from 50 μL of plasma using the TaqMan ABC miRNA Purification Kit (ThermoFisher). The synthetic microRNA ath-miR159a from Arabidopsis thaliana was used as a spiked-in control (single strand RNA, sequence: UUUGGAUUGAAGGGAGCUCUA (SEQ ID NO: 2)). Briefly, 100 ul ABC Buffer were added into 50 ul plasma, vortexed for 30 seconds to mix, then centrifuged briefly. 24, of 1 nM external control miRNA (ath-miR159a) was added into the prepared sample(s), vortexed to mix, centrifuged briefly, and then miRNA was purified according to the manufacturer's instructions. Finally, the microRNA was eluted in 100 μl Dnase and RNase free water.

For reverse transcription and preamplification, the total microRNA was condensed from 100 μl to 20 μl using a vacuum centrifuge condenser (Speed Vac Plus, SC110A, Savant) at low speed in room temperature. The cDNA was synthesized using TaqMan microRNA reverse transcription kit (Cat #: 4366596, ThermoFisher) according to the manufacturer's protocol. The RT product was preamplified with TaqMan PreAmp Master Mix (cat #4391128, ThermoFisher) and Megaplex PreAmp Primers (Cat #: 4399233 and 4444303, ThermoFisher) according to the manufacturer's protocol. The primers and probes used, and the miRNA sequences detected are set forth in Table 1, where “FAM” signifies the fluorescent dye fluorescein and “MGB” signifies a minor groove binder molecule.

TABLE 1
Primers and probes used to detect miRNA

| Name | | Sequence |
|---|---|---|
| miRNA | ath- | 5-UUUGGAUUGAAGGGAGCUCUA-3 (SEQ ID NO: 2) |
| | miR159a | |
| RT | 159aRT | 5-GTCGTATCCAGTGCAGGGTCCGAGGTATTCGCACTGGATACGACTAGAG-3 |
| primer | | (SEQ ID NO: 3) |
| Probe | 159aP | 5-FAM-CTGGATACGACTAGAGC-MGB-3 (SEQ ID NO: 4) |
| qPCR | 159aF | 5-GCCGTTTGGATTGAAGGGAGC-3 (SEQ ID NO: 5) |
| 5p | | |
| primer | | |
| qPCR | UPR | 5-GTGCAGGGTCCGAGGT-3 (SEQ ID NO: 6) |
| 3p | | |
| primer | | |
| miRNA | hsa- | 5-UGUAGUGUUUCCUACUUUAUGGA-3 (SEQ ID NO: 7) |
| | miR- | |
| | 142-3p | |
| RT | 142- | 5- |
| primer | 3pRT | GTCGTATCCAGTGCAGGGTCCGAGGTATTCGCACTGGATACGACTCCATAA-3 |
| | | (SEQ ID NO: 8) |
| Probe | 142-3pP | 5-FAM-CTGGATACGACTCCATAA-MGB-3 (SEQ ID NO: 8) |
| qPCR | 142-3pF | 5-CGCCGCTGTAGTGTTTCCTACTTT-3 (SEQ ID NO: 9) |
| 5p | | |
| primer | | |
| qPCR | UPR | 5-GTGCAGGGTCCGAGGT-3 (SEQ ID NO: 10) |
| 3p | | |
| primer | | |

To conduct openarray realtime PCR, the preamplification reaction product was diluted at 1:10 with 0.1×TE pH8.0. Forty μL of diluted preamplified pool A (or B) mixes were mixed with 40 μL 2×PCR master mix. Using an automated system, 5 μL of each pool was placed into 8 wells with an Xstream pipette (Eppendorf), and the openarray was covered with paraffin oil. The array was run with quantstudio 12 k flex (ThermoFisher).

To validate the specificity of microRNA PCR product from Taqman assay, the PCR product was cloned using a TA cloning kit (Cat #231124, Qiagen) and transformed into complement DH5a E. coli (Cat #C2987H, NEB). Plasmids from 20 clones from each miRNA were sequenced by Duke University DNA analysis facility. The DNA sequence alignments were performed by DNASTAR Lasergene Structural Biology Suite (DNASTAR).

### Example 2

### Profiling for Candidate miRNAs Using a High Throughput Platform

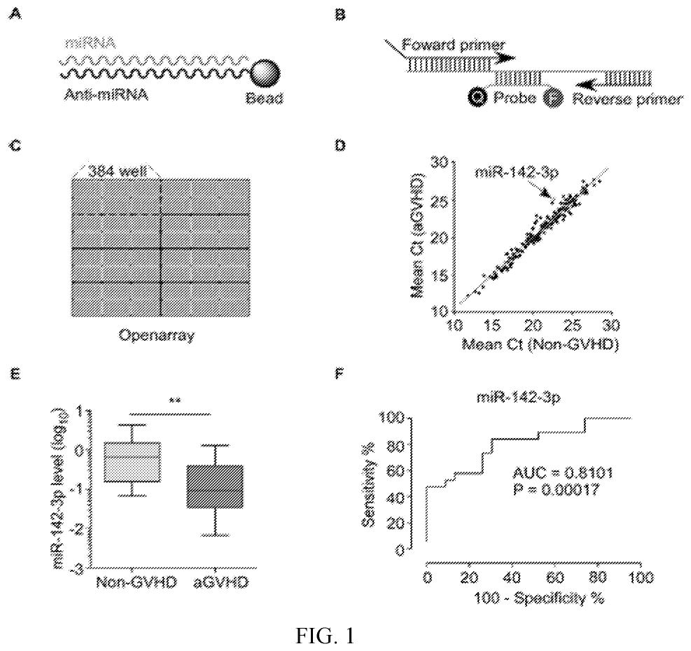

A microRNA sequence specific anti-miRNA bead capture method was used to purify microRNA from plasma (FIG. 1A). RT-PCR inhibitors and degraded RNA fragments commonly found in blood-related samples were washed away from the final elution. In addition, a probe based realtime PCR method (FIG. 1B) and openarray PCR chip (FIG. 1C) was used to improve the specificity and throughput. The platform was validated by sequencing five random microRNA PCR products from plasma microRNAs from a healthy donor. The sequencing results suggest that the platform could effectively and specifically detect microRNA from plasma samples (92%<specificity≤100%). The microRNA profiling was performed with the platform using plasma from 19 patients who had developed GVHD and 23 time-point matched non-GVHD subjects.

A majority of the plasma microRNAs showed similar expression patterns between disease and non-disease (FIG. 1D). Generalized linear models were used to analyze microRNAs that could distinguish aGVHD from non-GVHD.

The results showed that miRNA-142-3p was differentially expressed in plasma from aGVHD patients compared to that from non-GVHD patients. The expression levels of miR-142-3p were further validated in an independent openarray using another cohort of plasma samples from Dana-Farber Cancer Institute. The expression level of miR-142-3p was significantly lower in the plasma from aGVHD compared to that from non-GVHD (FIG. 1D, 1E). The AUC analysis indicated a high sensitivity and specificity of miR-142-3p in detecting aGVHD (FIG. 1F).

### Example 3

### Donor and Recipient Plasma miR-142-3p Ratio is Associated with aGVHD Development

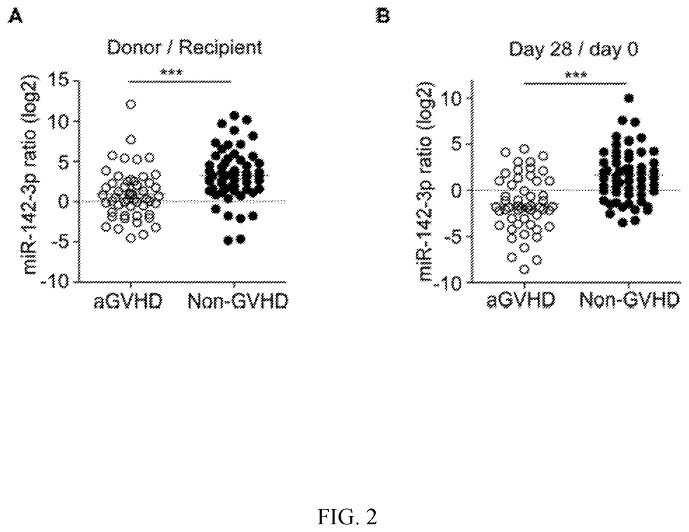

The expression level of miR-142-3p was determined to be lower in the plasma from aGVHD patients (FIG. 1D, 1E). The ratio of plasma miR-142-3p between donor and recipient was 4-fold lower on average in the aGVHD group compared to that from non-GVHD group. The expression level of miR-142-3p was also determined using plasma samples obtained on day 0 and day 28 after BMT. In both cases, the low ratio of miR-142-3p level was associated with aGVHD in the plasma from before BMT (FIG. 2A). In addition, the miR-142-3p ratio between day 0 and day 28 after BMT displayed similar pattern between aGVHD and non-GVHD groups (FIG. 2B). These data indicate that low miR-142-3p level in the donor plasma is a risk factor for the donor selection and develop of aGVHD after BMT. Moreover, the plasma miR-142-3p level in the recipient after BMT can be used to monitor the development of aGVHD.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

In case of conflict, the present specification, including definitions, will control. One skilled in the art will readily appreciate that the present invention is well adapted to carry out the objects and obtain the ends and advantages mentioned, as well as those inherent therein. The present disclosure described herein are presently representative of preferred aspects, are exemplary, and are not intended as limitations on the scope of the invention. Changes therein and other uses will occur to those skilled in the art which are encompassed within the spirit of the invention as defined by the scope of the claims.

We claim:

1. A method comprising:
(a) obtaining a first biological sample from a first subject at risk of GVHD, and
(b) detecting a level of miR-142-3p in the sample.

2. The method of claim 1, wherein the sample has a decreased level of miR-142-3p as compared to a second biological sample obtained from a second subject not at risk of GVHD.

3. The method of claim 1 or 2, wherein the first subject is in need of a bone marrow stem cell transplant.

4. The method of claim 2 or 3, further comprising comparing the level of miR-142-3p in the sample obtained from the first subject with a level of miR-142-3p obtained from the second subject.

5. The method of any one of claims 1-4, further comprising determining a risk of, prognosis of, or diagnosis of GVHD in the first subject.

6. The method of any one of claims 1-5, further comprising performing a stem cell transplant on the first subject.

7. A method for selecting a stem cell transplant donor, comprising:
(a) obtaining a first biological sample from a first subject and a second biological sample from a second subject,
(b) detecting a level of miR-142-3p in the first biological sample and the second biological sample;
(c) determining a ratio of (i) the level of miR-142-3p in the second biological sample to (ii) the level of miR-142-3p in the first biological sample; and
(d) determining the likelihood of the first subject will develop GVHD based on the ratio.

8. The method of claim 7, further comprising selecting a transplant donor based on the ratio.

9. The method of claim 7, further comprising not selecting a transplant donor based on the ratio.

10. The method of claim 7, further comprising transplanting stem cells from the second subject to the first subject if the ratio of (i):(ii) is at least about 2:1.

11. The method of claim 10, wherein the ratio is about 2.5:1, about 3:1, about 3.5:1, about 4:1, about 4.5:1, or about 5:1.

12. The method of claim 7, further comprising
obtaining one or more additional biological samples from one or more additional subjects, wherein the ratio of (i) the level of miR-142-3p in the second biological sample to (ii) the level of miR-142-3p in the first biological sample is higher than the ratio of (i) the level of miR-142-3p in the one or more additional biological samples to (ii) the level of miR-142-3p in the first biological sample; and
transplanting stem cells from the second subject to the first subject.

13. The method of any one of claims 2-12, wherein the first subject and the second subject are HLA matched.

14. The method of any one of claims 2-12, wherein the first subject and the second subject are HLA mismatched.

15. A method for determining whether a subject will develop GVHD, comprising:
(a) obtaining a first biological sample from a first subject that has undergone a stem cell transplant;
(b) detecting a level of miR-142-3p in the first biological sample;
(c) obtaining a second biological sample from the first subject, wherein the second biological sample is obtained from the subject after the first biological sample is obtained from the subject;
(d) determining a level of miR-142-3p in the second biological sample;
(e) determining a ratio of (i) the level of miR-142-3p in the second biological sample to (ii) the level of miR-142-3p in the first biological sample; and
(f) determining the likelihood the first subject will develop GVHD based on the ratio.

16. The method of claim 15, further comprising treating the subject for GVHD.

17. The method of claim 16, wherein the treatment comprises administering one or more of an immunosuppressive drug, a chemotherapy, a steroid, an antifungal agent, and antiviral agent, or an antibiotic.

18. The method of claim 17, wherein:
the immunosuppressive drug comprises one or more of cyclosporine, tacrolimus, methotrexate, sirolimus, mycophenolic acid, and rutiximab;
the chemotherapy comprises methotrexate;
the steroid comprises prednisone or methylprednisolone;
the antifungal agent comprises posaconazole;
the antiviral agent comprises acyclovir or valacyclovir; and
the antibiotic comprises sulfamethoxazole.

19. A method for determining the efficacy of a GVHD treatment, comprising:
(a) obtaining a first biological sample from a first subject that has been treated with an anti-GVHD therapy;
(b) detecting a level of miR-142-3p in the first biological sample;
(c) obtaining a second biological sample from the first subject, wherein the second biological sample is obtained from the subject after the first biological sample is obtained from the subject;
(d) determining a level of miR-142-3p in the second biological sample;
(e) determining a ratio of (i) the level of miR-142-3p in the second biological sample to (ii) the level of miR-142-3p in the first biological sample; and
(f) determining the efficacy of the anti-GVHD therapy based on the ratio.

20. The method of claim 19, further altering treatment of the GVHD if the ratio is below about 2:1.

21. The method of claim 19 or 20, wherein the anti-GVHD therapy comprises one or more of an immunosuppressive drug, a chemotherapy, a steroid, an antifungal agent, and antiviral agent, or an antibiotic.

22. The method of claim 21, wherein:
the immunosuppressive drug comprises one or more of cyclosporine, tacrolimus, methotrexate, sirolimus, mycophenolic acid, and rutiximab;
the chemotherapy comprises methotrexate;
the steroid comprises prednisone or methylprednisolone;
the antifungal agent comprises posaconazole;
the antiviral agent comprises acyclovir or valacyclovir; and
the antibiotic comprises sulfamethoxazole.

23. The method of any one of claims 1-22, wherein the first subject and/or the second subject is a mammal.

24. The method of claim 23, wherein the mammal is a human.

25. The method of any one of claims 1-24, wherein the first biological sample and/or the second biological sample is selected from the group consisting of tissues, cells, biopsies, blood, lymph, serum, plasma, urine, saliva, mucus, and tears.

26. The method according to claim 25, in which the sample comprises blood.

27. The method according to claim 25, in which the sample comprises plasma.

28. A composition for conducting the method of any one of claims 1-27, the composition comprising:
(a) a probe array for determining a level of miR-142-3p in a biological sample, the array comprising of a plurality of probes that hybridizes to the miR-142-3p; or
(b) a kit for determining a level of miR-142-3p in a biological sample, comprising the probe array of and instructions for carrying out the determination of miR-142-3p expression level in the biological sample.

29. The composition according to claim 28, in which the probe array of further comprises a solid support with the plurality of probes attached thereto.
